(19) United States
(12) Patent Application Publication
(10) Pub. No.: US 2026/0326716 A1
(43) Pub. Date: Oct. 22, 2026

(54) METHOD FOR DRIVING APPROVAL OF A VEHICLE FOR A DRIVER

(71) Applicant: MERCEDES-BENZ GROUP AG, Stuttgart (DE)

(72) Inventors: Friedrich Emanuel HUST, Esslingen (DE); Eric Rainer GRÜBNER, München (DE); Thomas BACKES, Kirchheim unter Teck (DE)

(21) Appl. No.: 19/109,812

(22) PCT Filed: Jul. 11, 2023
(86) PCT No.: PCT/EP2023/069201

§ 371 Date: Mar. 7, 2025

(30) Foreign Application Priority Data
Sep. 9, 2022 (DE) 10 2022 003 314.3

(51) Int. Cl. B60R 25/25; B60R 25/04

(52) U.S. Cl. CPC B60R 25/25; B60R 25/04

(57) ABSTRACT

A control device is used for driving approval of a vehicle for a driver. When contact with the steering wheel is detected an identification process of the current driver takes place. When the current driver is authorized to drive, a driving approval is granted for a time interval. The engine is only started when the steering wheel is contacted within the time interval.

### BACKGROUND AND SUMMARY OF THE INVENTION

Exemplary embodiments of the invention relate to a method for driving approval of a vehicle for a driver by means of a control device, as well as to the control device for performing the method.

It is already known from the prior art to record data via a vehicle steering wheel. U.S. Pat. No. 8,725,230 B2 describes a steering wheel for a vehicle having a sensor arrangement that can measure a biological parameter of the driver of the vehicle. DE 10 2020 005758 A1 describes a method for driving approval of a vehicle for a driver. In this case, the driver is authorized after touching the steering wheel of the motor vehicle and subsequently a timer with a specified duration is started. If the driver confirms their driving intent within the specified duration, the vehicle is started. The driver can confirm their driving intent by actuating the brake pedal and/or the transmission lever of the motor vehicle, for example. Consequently, systems for recording and evaluating signals from the brake pedal and/or transmission lever of the motor vehicle have to be provided in the vehicle. This causes additional costs and additional control effort.

U.S. Pat. No. 6,898,299 B1 discloses a method and an apparatus for authenticating a particular person. In this case, unique internal electric, magnetic or acoustic properties of several different body parts of this person can be determined and based thereon, a current biometric signature of this person is created. The biometric signature can then be used to identify the person.

DE 10 2017 211 545 A1 discloses an apparatus and a method for identifying whether a driver has taken their hands away from the steering wheel. In this method, a reference torque signal and a measured torque signal present in the case of a reference oscillation of the steering wheel are measured and evaluated.

DE 10 2007 005 627 A1 discloses a system for providing vehicle guest service information. In this case, biological characteristics of a user can be determined with the system. The system can support the use of the vehicle or entertain the user based on the biological characteristics. The characteristics can be stored in the system and retrieved.

DE 11 2020 003 270 T5 discloses an apparatus and a method for recording the position of hands of a driver on the steering wheel. For this purpose, two independent impedance measurements are carried out by means of two electrodes and the position of the hands of the driver is calculated or determined therefrom.

Therefore, exemplary embodiments of the invention are directed to an improved or at least alternative embodiment for a method of the generic type, by which the described disadvantages are overcome, as well as to a control arrangement for performing the method.

The present invention is based on the general idea of controlling a start-stop function in a vehicle by means of touching the steering wheel.

The method according to the invention is provided or designed for driving approval of a vehicle for a driver by means of a control device. In the method, the control device identifies a current driver when it detects contact with a steering wheel of the vehicle by the current driver. When the control device identifies the current driver as being an authorized driver, the control device grants driving approval for a predefined time interval. If the control device detects that the driver, identified as authorized, makes contact with the steering wheel of the vehicle in the specified time interval, it starts the engine of the vehicle. If the control device does not detect that the driver, identified as authorized, makes contact with the steering wheel of the vehicle in the specified time interval, it does not start the engine of the vehicle.

In the present invention, the terms “the authorized driver” and “the current driver” and “the driver identified as authorized” and “the driver identified as unauthorized” differ from each other. The authorized driver is a “virtual” driver characterized by a user profile. The user profile is—as described in more detail below—created in advance and stored. The current driver is the driver who is currently located at the steering wheel of the vehicle. The current driver can be authorized or unauthorized to drive. When the current driver is authorized to drive, they are referred to as the driver identified as authorized. When the current driver is not authorized to drive, they are referred to as the driver identified as unauthorized.

In the method, the start-stop function of the vehicle can be accessed directly. In this case, the driver identified as authorized can start the engine of the vehicle by placing their hands on the steering wheel and cannot start the engine by taking their hands away from the steering wheel. The existing start-stop systems can be expanded with a comfort function or replaced as far as possible by means of the method according to the invention. Additionally, the starting process of the vehicle can be facilitated as a result. Compared to conventional methods or to conventional vehicles, the recording and evaluation of signals from the brake pedal and/or the transmission lever of the motor vehicle can also be omitted. Therefore, additional costs and additional control efforts can be reduced.

After driving approval of the vehicle, the control device can also load the individual driver configurations of the driver identified as authorized. The individual driver configurations can, for example, comprise a seat setting and/or a mirror setting and/or driving dynamics and/or a valet service and/or driving space initiation.

When the control device does not identify the current driver as the authorized driver, the control device can only grant driving approval to a limited extent or not at all. The limited driving approval can, for example, be granted when a valet service is approved for the vehicle. Since the driving approval of the vehicle is carried out when the vehicle is stationary and before departure, an erroneous reaction by the driver cannot lead to any safety-critical events. Additionally, the control device can also initiate a security measure. The security measure can be implemented by making an entry in an error memory—a so-called security event log—and/or by deactivating the vehicle and/or by contacting a security system—a so-called incident response system—and/or by an acoustic and/or visual warning inside and/or outside the vehicle.

In order to be able to identify the current driver, the control device can determine once in advance an impedance of the authorized driver via their hands on the steering wheel of the vehicle. Then, when identifying the current driver, the control device can determine an impedance of the current driver via their hands on the steering wheel of the vehicle. After that, the control device can determine a deviation between the impedance of the current driver and the previously determined impedance of the authorized driver. When the deviation is inside a specified tolerance range, the control device can identify the current driver as the authorized driver. When, however, the deviation is outside the specified tolerance range, the control device can identify the current driver as the unauthorized driver. The tolerance range can be strictly predefined.

It should be understood that the control device can also determine the impedance of several authorized drivers in advance. Then, when the current driver is being identified, the control device can review user profiles of every authorized driver until the current driver is or is not identified as the authorized driver. The control device can review the corresponding user profiles one after the other or start the identification process with the user profile of the last driver.

Recording the impedance of human skin can take place in a manner known to the person skilled in the art. For this purpose, the control device can have a measuring unit with a measuring circuit and a measuring surface. The measuring surface can be arranged on the steering wheel of the vehicle such that it can be gripped or contacted by the driver and can be electrically connected to the measuring circuit. Two electrodes can be provided in the measuring surface, to which the measuring unit imparts a current or a voltage and records the voltage response or current response. If the hands of the driver are on the measuring surface, the impedance of their skin can be measured. The measured impedance depends on the frequency of the imparted current or voltage and is typically between 1.22 kHz and 1 MHz. It is understood that the impedance is the so-called electrochemical impedance.

Overall, the driver can control the vehicle by placing their hands on the steering wheel. By determining the impedance on the steering wheel, contact with the steering wheel can be detected, the identification of the current driver can be carried out and the vehicle started. Therefore, the starting process of the vehicle can be simplified.

The control device can record at least one surroundings parameter when determining the impedance of the current driver and/or when determining the impedance of the authorized driver in advance and can determine the impedance of the current driver and/or the previously determined impedance of the authorized driver taking into consideration the at least one surroundings parameter. For this purpose, a surroundings-dependent tolerance range can be defined for the impedance. Since the impedance depends on the surroundings parameters, incorrect identification of the current driver can be avoided. The surroundings parameter can be the moisture and/or the temperature. The change in the impedance due to moisture can be compensated for via a sweat pore model. The change in the impedance due temperature can be compensated for via the Arrhenius equation.

The following is advantageous: In order to keep the temperature at the electrodes and thus for the driver as constant as possible, it is advantageous to integrate effective temperature-regulating elements into the electrodes, in order to guarantee a uniform temperature (even over several measurement cycles). These can be both cooling elements as well as heating elements, which function in the body temperature range at approx. 37° C.

The control device can parameterize the previously determined impedance of the authorized driver by means of a mathematical model and thereby determine at least one parameter specific to the authorized driver. Analogously, the control device can also parameterize the impedance of the current driver by means of the mathematical model and thereby determine at least one parameter specific to the current driver. Subsequently, the control device can compare the respective parameter specific to the current driver and the respective parameter specific to the authorized driver when determining the deviation between the impedances. In other words, the deviation between the parameters determined with the model is determined or calculated when determining the deviation between the impedances.

The same model is used for the intended purpose when determining the specific parameters. The respective model reproduces the impedance of human skin via the respective specific parameters. The respective specific parameters describe characteristic properties of the skin and differ accordingly from person to person. These parameters can therefore be used to identify the person. The models for parameterizing the impedance of human skin are already known by the person skilled in the art and are not described further here. By way of example, the human skin can be described with the model from Tregear or the model from Montague or with the CPE-based model. Furthermore, the human skin can be approximated as a distribution of Debye-relaxation equations and a fractal expression. This model is more often mentioned as a model of distribution of relaxation times. The parameterization per se can take place with the method of least square fit or with the method of particle swarm optimization or with similar methods.

Determining the impedance of the authorized driver in advance takes place temporally before identifying the current driver. Determining the impedance of the authorized driver in advance can take place once and independently of time before identifying the current driver. In other words, the impedance of the authorized driver can take place once and several hours or days or weeks before first identifying the current driver. Determining the impedance of the authorized driver in advance can be fundamentally viewed as a learning procedure for the control device. Determining the impedance of the authorized driver in advance can, for example, take place when the control device is started up or before the first journey. In principle, however, it is conceivable that the renewed advance determination of the impedance of the authorized driver or the initial advance determination of the impedance of another authorized driver could also take place later. The advance determination of the impedance or the learning procedure can be manually initiated, for example, in a further step of the method at any time.

The control device can create a user profile for the authorized driver and store it in a non-volatile memory in a security module. The user profile can comprise at least the previously determined impedance of the authorized driver and/or the respective parameter specific to the authorized driver. The security module can be a specially protected module such as a so-called security IC—for example NXP phantom NCJ38A0HN or similar. The security module can also be a hardware security module of a microcontroller—for example Infineon AURIX. The security module can be an integral and hard-to-reach control device of an electronic ignition lock. The user profile can be protected from misuse—such as for example from unauthorized deletion—due to the non-volatile memory.

The control device can create a personal identification number for the created user profile and can store the personal identification number in a non-volatile memory in the security module together with the user profile. The control device only allows the stored user profile to be changed after input of the correct personal identification number. The user profile can be protected from misuse—such as for example from unauthorized deletion—due to the personal identification number or PIN. A request or input of the personal identification number can, for example, take place via a monitor of the control device or via a mobile end device. In particular, the personal identification number is required when creating and deleting the user profile.

The control device can determine the impedance of the current driver—as already described above-with a measuring unit. The control device can determine the deviation and compare the deviation with the specified tolerance range in a security module physically separate from the measuring unit. The security module can be designed as described above.

In order to increase the security of the method, the control device can determine an impedance of an identification circuit with the impedance of the current driver or separately therefrom. Then, the control device can compare a deviation between the currently determined impedance of the identification circuit and a previously determined impedance of the identification circuit. If the deviation is within a specified tolerance range, it is assumed that the identification circuit has not been tampered with, and driving approval is granted. If the deviation is outside the specified tolerance range, it is assumed that the identification circuit has been tampered with, and driving approval is only granted to a limited extent, or not at all.

The identification circuit can be a part of the above-described measuring unit of the security device and can be measured alone or in series connection with the measuring surface. For this purpose, the measuring unit can have a correspondingly connected switch. In one position of the switch, the measuring surface on the steering wheel of the vehicle and the identification circuit can be connected in series to each other and to the measuring circuit, and in the other position of the switch, only the identification circuit can be connected in series to the measuring circuit. The switch can be a transistor or a MOSFET or a relay or another electrical switch. As the change in the impedance recorded by the measuring circuit on the measuring surface is small, the relay can be particularly suitable for this.

The impedance of the identification circuit can be parameterized by means of a mathematical model—for example by means of distribution of Debye relaxation equations—and the identification features of the identification circuit can be determined from the model. This makes use of the fact that the same switching circuits or the switching circuits with the same elements have differing identification features and therefore can be clearly differentiated from each other. The identification circuit can have at least two elements—for example, a coil and/or an ohmic resistor and/or a capacitor—which can be connected arbitrarily to each other. In particular, the identification circuit can have an ohmic resistor and a capacitor which together can represent distribution of Debye relaxation equations and can be identified particularly well. The elements can, in particular, be selected so that the distribution of Debye relaxation equations of these elements is characterized with frequencies, which are two to three decades before and/or after the typical values of the impedance of the current driver.

The impedance or the unique identification features of the identification circuit can even be determined in advance in the factory and stored in a non-volatile memory in the control device—for example in the above-described security module.

The identification circuit is designed according to the invention to be physically hard to reach, for example encapsulated. According to the invention, the identification circuit is installed in the steering wheel of the vehicle. Then, according to the invention, both tampering with the identification circuit as well as the swapping of the steering wheel with the identification circuit is recognized by means of the identification circuit.

After the driving approval, according to the invention, the control device continually determines in a driving mode of the vehicle the impedance of the driver identified as authorized. Then, the user profile of the authorized driver can be continually adapted according to the determined impedance. Therefore, a change over time in the impedance of the authorized driver can be taken into account and the robustness of identification can be increased.

The invention also relates to a control device for a vehicle, wherein the control device is designed to perform the above-described method. The control device can have a measuring unit for determining the impedance. The measuring unit can have a measuring circuit and a measuring surface. Additionally, the measuring unit can also have an identification circuit and an electrical switch. The switch can be switched in such a way that the measuring surface and the identification circuit are connected in series to each other and to the measuring circuit or just the identification circuit is connected in series to the measuring circuit. The control device can have a security module. In order to avoid repetitions, please refer to the above embodiments.

Further important features and advantages of the invention result from the dependent claims, the drawings and the associated figure description with reference to the drawings.

It is understood that the aforementioned features and the features to be explained in the following can not only be used in the respectively specified combination, but also in other combinations or in isolation, without leaving the scope of the present invention.

Preferred exemplary embodiments of the invention are represented in the drawings and are explained in more detail in the following description, wherein the same reference signs refer to identical or similar or functionally identical components.

### BRIEF DESCRIPTION OF THE DRAWING FIGURES

In the drawings:

FIG. 1 schematically shows a view of a vehicle having a control device according to the invention;

FIG. 2 schematically shows a circuit diagram of a measuring unit of the control device according to the invention;

FIG. 3 schematically shows a diagram of a learning procedure in a method according to the invention; and

FIG. 4 schematically shows a general diagram of the method according to the invention.

### DETAILED DESCRIPTION

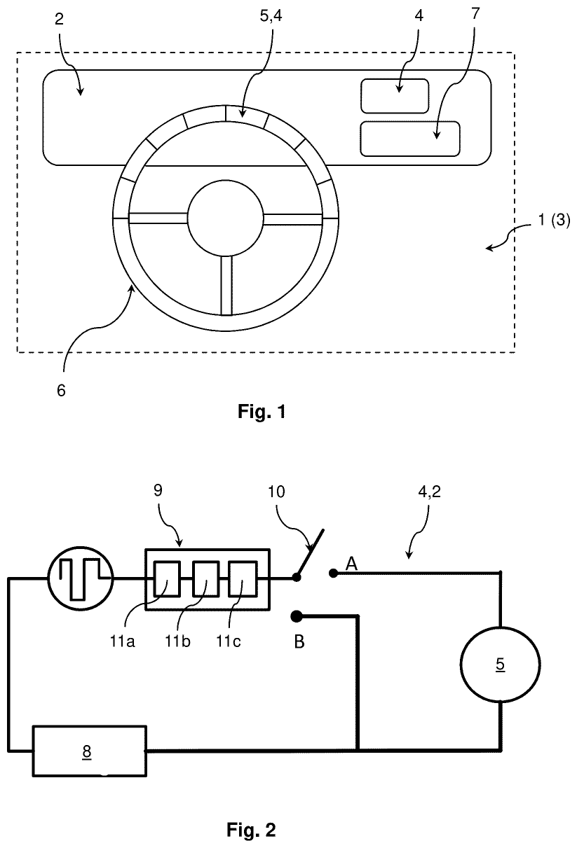

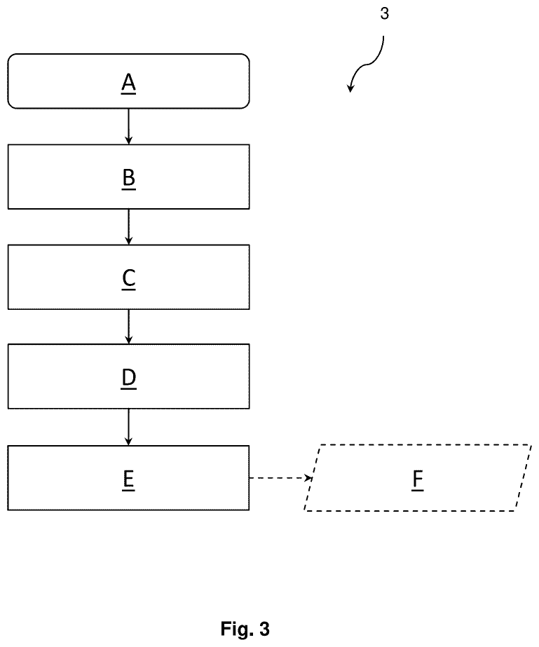

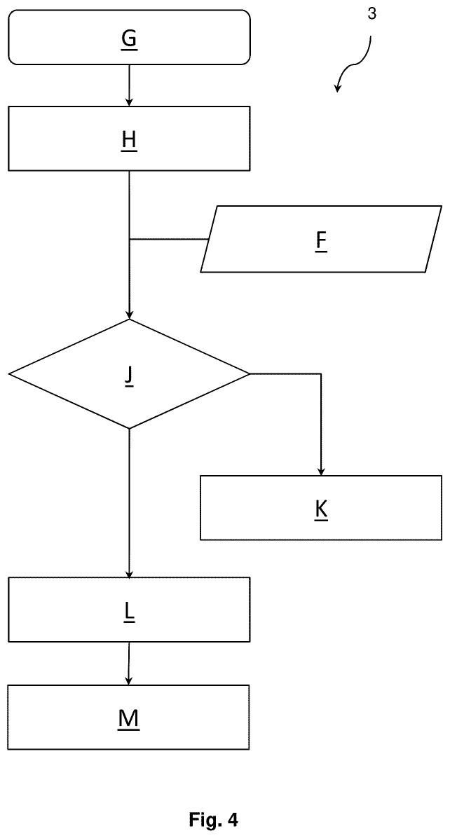

FIG. 1 shows a view of a vehicle 1 having a control device 2 according to the invention. The control device 2 is designed to perform a method 3 according to the invention for driving approval of the vehicle 1 for a driver. The method 3 is explained in more detail in the following using FIG. 3 and FIG. 4. The control device 2 comprises a measuring unit 4 having a measuring surface 5 arranged on a steering wheel 6 of the vehicle 1. If the hands of the driver are located on the measuring surface 5 of the measuring unit, the measuring unit 4 can detect that contact is made with the steering wheel 6 and record an impedance of the driver. The measuring unit 4 is explained in more detail in the following using FIG. 2. The control device 2 also comprises a security module 7, which is difficult to reach and tamper-proof. The security module 7 is, in particular, designed for storing and processing security-relevant data in the method 3.

FIG. 2 shows a circuit diagram of the measuring unit 4 of the control device 2 according to the invention. As already described above, the measuring unit 4 has the measuring surface 5 for determining the impedance of the driver. Furthermore, the measuring unit 4 has a measuring circuit 8 and an identification circuit 9. Additionally, an electrical switch 10 is provided in the measuring unit 4, which, for example, is a transistor or a MOSFET or a relay or another electrical switch. In a position A, the switch 10 connects the measuring surface 5, the measuring circuit 8 and the identification circuit 9 in series. In a position B, the switch 10 connects the measuring circuit 8 and the identification circuit 9 in series. The measuring surface 5 remains disconnected in this case.

Several elements—for example, an ohmic resistor and/or a coil and/or a capacitor—can be connected arbitrarily to each other in the identification circuit 9. Therefore, the identification circuit 9 has unique identification features 11a, 11b, 11c and can be uniquely identified. These identification features 11a, 11b, 11c can be determined by means of a model—for example by means of distributing Debye relaxation equations—from the impedance of the identification circuit 9. The identification circuit 9 can be designed as hard to reach from the outside, for example encapsulated.

FIG. 3 shows a diagram of the advance determination of an impedance of the authorized driver or a learning procedure of the control device 2 in the method 3 according to the invention. In step A, the learning procedure is started. In step B, an impedance F of the authorized driver is measured by means of the measuring unit 4. Additionally, the surroundings parameters, such as moisture and temperature for example, can be recorded and the influence thereof on the measured impedance F of the authorized driver is eliminated from calculations. In step C, the measured impedance F of the authorized driver is parameterized in a mathematical model and at least one parameter specific to the authorized driver is determined. In step D, a user profile is created for the authorized driver and a personal identification number is created for the user profile. In step E, the user profile is stored in a non-volatile memory with the impedance F or with the respective specific parameter of the impedance F and the personal identification number in the security module 7 of the control device 2. The user profile can only be changed after input of the correct identification number.

FIG. 4 shows a general diagram of the method 3 according to the invention. In step G, the method 3 is started by hands being placed on the steering wheel 6 of the vehicle 1, for example. In step H, an impedance of the current driver is measured by means of the measuring unit 4. Additionally, the surroundings parameters, such as moisture and temperature, can be recorded and the influence thereof on the measured impedance of the current driver can be taken into account. In step J, the measured impedance of the current driver is compared with the previously determined impedance F of the authorized driver. If the deviation between the measured impedance of the current driver and the previously determined impedance F of the authorized driver is outside of a tolerance range, in step K the current driver is recognized as the unauthorized driver and driving approval is only granted to a limited extent or not at all. If the deviation between the measured impedance of the current driver and the previously determined impedance F of the authorized driver is inside a tolerance range, in step L the current driver is recognized as the authorized driver and driving approval is granted. In step J, several user profiles can also be checked one after the other or the measured impedance of the current driver can be compared to previously determined impedances F from several previously stored user profiles. Subsequently, in step M, the control device 2 can load the individual driver configurations—for example, a seat setting and/or a mirror setting and/or driving dynamics and/or a valet service and/or driving space initiation—of the driver identified as authorized.

In the method 3, the learning procedure or the advance determination of the impedance of the authorized driver in steps A to E according to FIG. 3 is carried out temporally before the further steps G to L according to FIG. 4. In other words, at least one user profile created in steps A to E according to FIG. 3 has to be present in the security module 7 in order to perform steps G to L according to FIG. 4. Further user profiles, however, can be created at any other time and stored in the security module 7.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-9. (canceled)

10. A method for driving approval of a vehicle for a driver by a control device, the method comprising:
identifying, by the control device, a current driver when the control device detects contact with a steering wheel of the vehicle by the current driver;
granting, by the control device responsive to determining that the current drive is an authorized driver, driving approval for a predefined time interval;
starting, by the control device, an engine of the vehicle responsive to detecting that the authorized driver contacts the steering wheel of the vehicle in the predefined time interval;
not starting, by the control device, the engine of the vehicle responsive to detecting that the authorized driver does not contact the steering wheel of the vehicle in the predefined time interval;
continually determining, by the control device after granting the driving approval, an impedance of the current driver identified as authorized and continually adapting a user profile of the authorized driver according to the determined impedance; and
recognizing, by the control device, that an identification circuit installed in a physically hard to reach place and in the steering wheel of the vehicle, the control device has been tampered with or the steering wheel with the identification circuit has been swapped.

11. The method of claim 10, further comprising:
determining, by the control device once prior to the identifying the current driver, an impedance of the authorized driver via the authorized driver's hands on the steering wheel of the vehicle,
wherein, when identifying the current driver, the control device determines an impedance of the current driver via the current driver's hands on the steering wheel of the vehicle,
wherein the control device determines a deviation between the impedance of the current driver and the impedance of the authorized driver determined once prior to the identifying the current driver,
wherein the control device identifies the current driver as the authorized driver when the deviation is within a specified tolerance range,
wherein the control device identifies the current driver as an unauthorized driver when the deviation is outside the specified tolerance range.

12. The method of claim 11, further comprising:
recording, by the control device, at least one surroundings parameter when determining the impedance of the current driver or when determining the impedance of the authorized driver determined once prior to the identifying the current driver; and
determining, by the control device, the impedance of the current driver or the impedance of the authorized driver determined once prior to the identifying the current driver taking into consideration the at least one surroundings parameter.

13. The method of claim 12, further comprising:
parameterizing, by the control device, the impedance of the of the authorized driver determined once prior to the identifying the current driver by a mathematical model to determine at least one parameter specific to the authorized driver;
parameterizing, by the control device, the impedance of the current driver by the mathematical model to determine at least one parameter specific to the current driver; and
comparing, by the control device, the parameter specific to the current driver and the parameter specific to the authorized driver when determining the deviation between the impedances.

14. The method of claim 11, further comprising:
creating, by the control device, a user profile for the authorized driver; and
storing, by the control device, the user profile for the authorized driver in a non-volatile memory in a security module,
wherein the user profile comprises at least the impedance of the of the authorized driver determined once prior to the identifying the current driver or the parameter specific to the authorized driver.

15. The method of claim 14, wherein the control device creates a personal identification number for the created user profile and stores the personal identification number in the non-volatile memory in the security module together with the user profile, and wherein the control device only allows the stored user profile to be changed after input of a personal identification number matching the created personal identification number.

16. The method of claim 11, wherein the control device determines the impedance of the current driver with a measuring unit, and wherein the control device determines the deviation and compares the deviation with the specified tolerance range in a security module physically separate from the measuring unit.

17. The method of claim 11, wherein the control device determines an impedance of the identification circuit with the impedance of the current driver or separately, wherein the control device determines a deviation between the currently determined impedance of the identification circuit and a previously determined impedance of the identification circuit, wherein the control device assumes that the identification circuit has not been tampered with and grants driving approval responsive to the deviation being within a specified tolerance range, and wherein the control device assumes that the identification circuit has been tampered with and only grants driving approval to a limited extent, or not at all, responsive to the deviation being outside the specified tolerance range.

18. A control device for a vehicle, the control device configured to:
identify a current driver when the control device detects contact with a steering wheel of the vehicle by the current driver;
grant, responsive to determining that the current drive is an authorized driver, driving approval for a predefined time interval;
start, an engine of the vehicle responsive to detecting that the authorized driver contacts the steering wheel of the vehicle in the predefined time interval;
not start the engine of the vehicle responsive to detecting that the authorized driver does not contact the steering wheel of the vehicle in the predefined time interval;
continually determine, after granting the driving approval, an impedance of the current driver identified as authorized and continually adapt a user profile of the authorized driver according to the determined impedance; and
recognize that an identification circuit installed in a physically hard to reach place and in the steering wheel of the vehicle, the control device has been tampered with or the steering wheel with the identification circuit has been swapped.
